(19) United States
(12) Patent Application Publication
(10) Pub. No.: US 2025/0386138 A1
(43) Pub. Date: Dec. 18, 2025

(54) CONTINUOUS POLYPHONIC AUDIO SYNTHESIZER FOR SURGICAL LOCALIZATION FEEDBACK

(71) Applicant: Stryker Corporation, Portage, MI (US)

(72) Inventors: Mark SEMPLE, Toronto (CA); Ananth RAVI, Toronto (CA); John DILLON, Mississauga (CA)

(73) Assignee: Stryker Corporation, Portage, MI (US)

(21) Appl. No.: 19/236,722

(22) Filed: Jun. 12, 2025

Related U.S. Application Data
(60) Provisional application No. 63/659,829, filed on Jun. 13, 2024.

(51) Int. Cl. H04R 3/04; H04R 3/02

(52) U.S. Cl. CPC H04R 3/04; H04R 3/02

(57) ABSTRACT

An exemplary method for providing feedback by a localization system comprises: obtaining location information of a marker, wherein the location information is generated by the localization system; generating a feedback signal comprising a plurality of audio tones, wherein each audio tone has a frequency different from the other audio tones of the plurality of audio tones, and wherein each audio tone is associated with a respective activation function of a plurality of activation functions, the respective activation function configured to modulate an amplitude of the respective audio tone based on the obtained location information of the marker; and providing the feedback signal to indicate the location information of the marker.

### CROSS-REFERENCE TO RELATED APPLICATION

This application claims the benefit of U.S. Provisional Application 63/659,829 filed on Jun. 13, 2024, the entire content of which is incorporated herein by reference for all purposes.

### FIELD OF INVENTION

The present disclosure relates generally to localization systems, and more specifically to feedback signals provided by localization systems.

### BACKGROUND

In many medical procedures, precise localization of an area of interest, such as a tumor or anatomical structure, can be critical. A marker (e.g., a magnetic marker) can be placed at or near the area of interest preoperatively or intraoperatively. During the medical procedure, a probe device of a localization system is used by the surgeon to locate the marker. As the probe device moves relative to the marker, the probe device can detect signals emitted or reflected by the marker. The localization system can provide real-time feedback signals (e.g., auditory, visual, tactile, or the like) to guide the surgeon. The feedback signal can change depending on the proximity of the probe device to the marker.

Current techniques for providing a feedback signal by the localization system suffer from several deficiencies. In some systems, the feedback signal is an audio signal with a frequency that can increase or decrease according to the distance between the marker and the probe device. However, these audio signals are deficient because they may produce unpleasant sounds and can be damaging to the speakers.

### BRIEF SUMMARY

This disclosure describes a novel digital synthesizer design for surgical localization feedback. Disclosed herein are systems, electronic devices, methods, non-transitory storage media, and apparatuses for providing feedback by a localization system. An exemplary system (e.g., one or more electronic devices) can obtain location information of a marker (e.g., a magnetic marker or any object that can cause a signal detectable by the localization system). The location information is indicative of targeting precision and can include any aspect of the location of the marker. In some examples, the location information can include an individual component of a 3D position or any combination of multiple components (e.g., relative to a probe device), a distance (e.g., between the marker and the probe device), angular data (e.g., alignment data relative to the probe device), or any combination thereof. The location information can be generated by the localization system, for example, based on measurement data (e.g., magnetic measurements) obtained by a detector of the localization system.

Based on the location information, the system can generate a feedback signal comprising a plurality of audio tones. Each audio tone has a frequency different from the other audio tones of the plurality of audio tones, and each audio tone is associated with a respective activation function of a plurality of activation functions. The respective activation function modulates an amplitude of the respective audio tone based on the obtained location information of the marker. The system can provide the feedback signal to indicate the location information of the marker, such that a surgeon can identify the location of interest more quickly and more accurately. The resulting feedback signal can be more pleasing to the ear, provide more nuanced feedback (e.g., more degrees of feedback), and be less damaging to the speakers.

Current techniques for providing a feedback signal by the localization system suffer from several deficiencies. In some systems, the feedback signal is a continuous audio signal with frequency inversely proportional to the distance between the marker and the probe device. As the probe device approaches or moves away from the marker, the audio signal frequency can continuously increase or decrease. The rapid increase to high pitches results in an unpleasant, shrill sound that is distracting and disruptive in the surgical environment. In some other systems, the feedback signal is a discontinuous audio signal with predefined frequency steps. Such feedback signal provides poor resolution of feedback, making it difficult for the surgeon to understand the immediate impact of smaller movements. Further, the sudden jumps in frequencies can be unnatural and jarring, and the rapid switching between frequency steps may put stress on speakers and lead to faster deterioration over time.

Examples of the present disclosure can provide several technical advantages. The techniques can produce a continuous polyphonic signal that is pleasing to the ear and provides nuanced feedback. In some examples, the techniques can produce a dynamic waveform with natural-sounding transitions between notes (for example, between emphasized notes). The resulting waveform is a continuous waveform with multiple audio tones blending into one another. In the continuous waveform, as one audio tone increases in magnitude, another audio tone simultaneously decreases in magnitude. At a given time point, multiple audio tones are played simultaneously but the audio tone most closely corresponding to the location information may outweigh the other audio tones in magnitude. Thus, there may be no abrupt steps in the signal and the resulting soundwave is more pleasing to the ear. The technique can be implemented with, for example, vectorized calculations, making it highly efficient and well-suited for real-time use. Because the techniques does not require retrieving sample audio data items, loading them, and mixing the sample audio data items to generate feedback signals, the techniques provide improved efficiency, fewer points of failure, more lightweight code, and reduced processor and memory usage. As a dynamic mixture of tones, the output waveform is less likely to cause speaker cone-damaging resonance effects than a comparable pure tone.

This design can be for surgical localization procedures in which a surgeon uses a detector probe to find an implanted marker target or marker. The detection system provides feedback signal to guide the surgeon towards their target.

An exemplary method for providing feedback by a localization system comprises: obtaining location information of a marker, wherein the location information is generated by the localization system; generating a feedback signal comprising a plurality of audio tones, wherein each audio tone has a frequency different from the other audio tones of the plurality of audio tones, and wherein each audio tone is associated with a respective activation function of a plurality of activation functions, the respective activation function configured to modulate an amplitude of the respective audio tone based on the obtained location information of the marker; and providing the feedback signal to indicate the location information of the marker.

In some examples, the localization system comprises a probe device comprising one or more magnetic sensors. The location information of the marker can be indicative of target precision of the probe device. The location information can comprise a component of a 3D position, a combination of multiple components of the 3D position, a distance, angular data, or any combination thereof.

The activation function of the plurality of activation functions may be a probability density function. The width of an activation function of the plurality of activation functions may be the same as or different from a width of another activation function of the plurality of activation functions. The peak amplitude of an activation function of the plurality of activation functions may be the same as or different from a peak amplitude of another activation function of the plurality of activation functions. In some examples, the center frequencies of the plurality of activation functions are configured to be evenly spaced or vary.

In some examples, providing the feedback signal comprises outputting the feedback signal via a speaker.

An exemplary system for providing feedback comprises: a localization system; and a processor in electronic communication with the localization system, wherein the processor is configured to: obtain location information of a marker, wherein the location information is generated by the localization system; generate a feedback signal comprising a plurality of audio tones, wherein each audio tone has a frequency different from the other audio tones of the plurality of audio tones, and wherein each audio tone is associated with a respective activation function of a plurality of activation functions, the respective activation function configured to modulate an amplitude of the respective audio tone based on the obtained location information of the marker; and provide the feedback signal to indicate the location information of the marker.

In some examples, the localization system comprises a probe device comprising one or more magnetic sensors. The location information of the marker can be indicative of target precision of the probe device. The location information can comprise a component of a 3D position, a combination of multiple components of the 3D position, a distance, angular data, or any combination thereof.

The activation function of the plurality of activation functions may be a probability density function. The width of an activation function of the plurality of activation functions may be the same as or different from a width of another activation function of the plurality of activation functions. The peak amplitude of an activation function of the plurality of activation functions may be the same as or different from a peak amplitude of another activation function of the plurality of activation functions. In some examples, the center frequencies of the plurality of activation functions are configured to be evenly spaced or vary.

In some examples, providing the feedback signal comprises outputting the feedback signal via a speaker.

An exemplary non-transitory computer-readable storage medium storing one or more programs for providing feedback by a localization system, the one or more programs comprising instructions, which when executed by one or more processors of an electronic device, cause the electronic device to perform any of the methods described herein and any combination of the methods.

### DESCRIPTION OF THE FIGURES

For a fuller understanding of the nature and objects of the disclosure, reference should be made to the following detailed description taken in conjunction with the accompanying drawings.

FIG. 1 is a functional illustration of synthesizer according to an embodiment of the present disclosure.

FIG. 2 illustrates activation functions of four audio tones distributed over a marker distance scale.

FIG. 3 illustrates a Gaussian probability density function.

FIG. 4 illustrates an exemplary method for providing feedback by a localization system, in accordance with some examples.

FIG. 5A illustrates an exemplary marker localization system, in accordance with some examples.

FIG. 5B depicts use of an exemplary marker localization system, in accordance with some examples.

FIG. 6A illustrates an exemplary marker localization system, in accordance with some examples.

FIG. 6B illustrates an exemplary marker localization system, in accordance with some examples.

FIG. 7 illustrates an example of a computing device, in accordance with some examples.

### DETAILED DESCRIPTION

This disclosure describes a novel digital synthesizer design for surgical localization feedback. Disclosed herein are systems, electronic devices, methods, non-transitory storage media, and apparatuses for providing feedback by a localization system. An exemplary system (e.g., one or more electronic devices) can obtain location information of a marker (e.g., a magnetic marker or any object that can cause a signal detectable by the localization system). The location information is indicative of targeting precision and can include any aspect of the location of the marker. In some examples, the location information can include an individual component of a 3D position or any combination of multiple components (e.g., relative to a probe device), a distance (e.g., between the marker and the probe device), angular data (e.g., alignment data relative to the probe device), or any combination thereof. The location information can be generated by the localization system, for example, based on measurement data (e.g., magnetic measurements) obtained by a detector of the localization system.

Based on the location information, the system can generate a feedback signal comprising a plurality of audio tones. Each audio tone has a frequency different from the other audio tones of the plurality of audio tones, and each audio tone is associated with a respective activation function of a plurality of activation functions. The respective activation function modulates an amplitude of the respective audio tone based on the obtained location information of the marker. The system can provide the feedback signal to indicate the location information of the marker, such that a surgeon can identify the location of interest more quickly and more accurately. The resulting feedback signal can be more pleasing to the ear, provide more nuanced feedback (e.g., more degrees of feedback), and be less damaging to the speakers.

Current techniques for providing a feedback signal by the localization system suffer from several deficiencies. In some systems, the feedback signal is a continuous audio signal with frequency inversely proportional to the distance between the marker and the probe device. As the probe device approaches or moves away from the marker, the audio signal frequency can continuously increase or decrease. The rapid increase to high pitches results in an unpleasant, shrill sound that is distracting and disruptive in the surgical environment. In some other systems, the feedback signal is a discontinuous audio signal with predefined frequency steps. Such feedback signal provides poor resolution of feedback, making it difficult for the surgeon to understand the immediate impact of smaller movements. Further, the sudden jumps in frequencies can be unnatural and jarring, and the rapid switching between frequency steps may put stress on speakers and lead to faster deterioration over time.

Examples of the present disclosure can provide several technical advantages. The techniques can produce a continuous polyphonic signal that is pleasing to the ear and provides nuanced feedback. In some examples, the techniques can produce a dynamic waveform with natural-sounding transitions between notes (for example, between emphasized notes). The resulting waveform is a continuous waveform with multiple audio tones blending into one another. In the continuous waveform, as one audio tone increases in magnitude, another audio tone simultaneously decreases in magnitude. At a given time point, multiple audio tones are played simultaneously but the audio tone most closely corresponding to the location information may outweigh the other audio tones in magnitude. Thus, there may be no abrupt steps in the signal and the resulting soundwave is more pleasing to the ear. The technique can be implemented with, for example, vectorized calculations, making it highly efficient and well-suited for real-time use. Because the techniques do not require retrieving sample audio data items, loading them, and mixing the sample audio data items to generate feedback signals, the techniques provide improved efficiency, fewer points of failure, more lightweight code, and reduced processor and memory usage. As a dynamic mixture of tones, the output waveform is less likely to cause speaker cone-damaging resonance effects than a comparable pure tone.

This design can be for surgical localization procedures in which a surgeon uses a detector probe to find an implanted marker target or marker. The detection system provides feedback signal to guide the surgeon towards their target.

In various embodiments, the synthesizer is considered “polyphonic” because it includes an array of tones (e.g., two or more) and at least a subset of the audio tones (in some cases, all of the audio tones) are played simultaneously. Each audio tone has a continuous activation function that describes its influence (e.g., amplitude, or relative loudness) for any input value.

This synthesizer design may produce a dynamic waveform with natural-sounding transitions between notes (for example, between emphasized notes). This technique can be implemented with, for example, vectorized calculations, making it highly efficient and well-suited to real-time use. Vectorized calculations (e.g., synthesizer populating an audio buffer) can be parallelized and thus involve light and fast computation, making it particularly advantageous for real-time processing. As a dynamic mixture of tones, the output waveform is less likely to cause speaker cone-damaging resonance effects than a comparable pure tone.

In an embodiment, the input signal (x) may be the marker's 3D positional coordinate relative to the detector. From this positional coordinate, the vector norm (i.e. distance) is easily determined. Some component of the marker's coordinate (e.g., depth, lateral offset, net distance, 5D pose, etc.) can be used to drive the activation functions of the sub-tones activation functions, which informs their relative amplitudes. In some examples, angular data (e.g., alignment data) can be provided as at least a part of the input signal (x). The sub-tones are summed together to produce the net waveform for output.

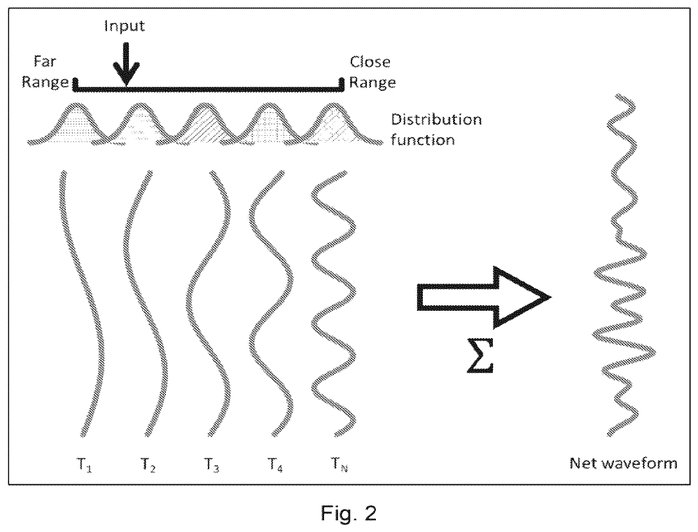

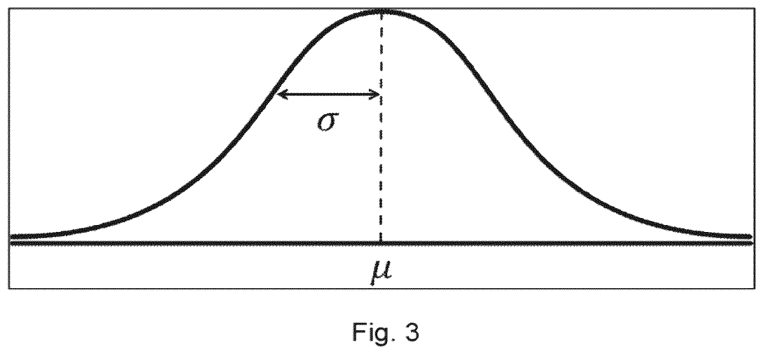

FIG. 2 illustrates five exemplary audio tones (sometimes referred to herein as “sub-tones”) which are evenly distributed across a marker distance scale from “far range” to “close range.” It can be seen that the activation function of each sub-tone will vary the amplitude (y-axis) of each subtone depending on the position along the scale (x-axis). The distributions of the various tones overlaps such that more than one tone will be audible at certain locations. In some embodiments, the distributions may have larger overlapping ranges than those depicted in FIG. 2 such that more than two sub-tones may be heard at certain marker locations. A combination of the sub-tones results in a net waveform which may be provided as a feedback signal. For example, the feedback signal may be provided to a speaker.

| | |
|---|---|
| Wₒᵤₜ | The net waveform to be played by the speakers. |
| x | The input signal, based on the location information (e.g., relative 3-D marker |
| | position with respect to the detector) of the marker. The location information is |
| | indicative of targeting precision and can include any aspect of the location of the |
| | marker. In some examples, the location information can include an individual |
| | component of a 3D position or any combination of multiple components (e.g., |
| | relative to a probe device), a distance (e.g., between the marker and the probe |
| | device), angular data (e.g., alignment data relative to the probe device), or any |
| | combination thereof. For example, it can be an individual component of the |
| | position, or the net distance (position vector norm). |
| {T} | The set of all sub-tones. More than one, but can be any positive integer. |
| T_{N} | Sub-tone N. A periodic function, for example: sine wave, square wave, triangle |
| | wave, sawtooth wave. |
| f_{N} | The root frequency (Hz) for sub-tone N. Helpful to be distinct from the other |
| | subtones. |
| A_{N}(x) | Continuous activation function for sub-tone N. This takes input x and provides the |
| | sub-tone's activation level. For example, a Gaussian probability distribution |
| | function. |
| | In the polyphonic synthesizer, all sub-tones can be played simultaneously. Each |
| | sub-tone's relative amplitude is driven by its activation level. The activation level |
| | can be determined with the probability density function (PDF). This PDF is a |
| | continuous function with a value for all real numbers. For example, a Normal |
| | distribution (Gaussian), lognormal, Laplace, etc. (See, e.g., FIG. 3) |
| α_{N} | Activation level for sub-tone N. This is the output of the continuous activation |
| | function. This scalar is used to set relative amplitude of this sub-tone. |
| μ_{N} | Centre location for sub-tone N. This property defines the input position at which |
| | this tone's influence is a maximum. The centers of the sub-tones are spaced over |
| | the detection range, either linearly or nonlinearly (e.g., quadratic, etc.) |
| σ_{N} | Spread for sub-tone N. This property defines the shape of the activation function |
| | and affects the blending of neighboring sub-tones. Narrow spread makes the notes |
| | more individually distinct, and a widespread makes the notes more blended |
| | together. |

Any of these properties can be pre-set constants or be dynamically driven by the input signal (linearly or nonlinearly).

In some embodiments, each activation function is a continuous function. The amplitude of each sub-tone may be calculated over the entire scale of the marker distance (although the resulting amplitude of an audio tone may be insufficient to drive a speaker using such audio tone at certain marker distances). The final feedback signal comprises a plurality of audio tones and thus is continuous across multiple activation functions.

### Example Embodiment

In an example embodiment, a processor (synthesizer) was initialized with 27 sub-tones (N=27), selected from the chromatic musical scale between 300 and 1400 Hz. The center frequencies of the sub-tones were evenly distributed over a scale of input signal representing a marker distance of between 50 and 0 mm. The width of each activation function was set to 2 mm. In this way, a sub-tone was audible for a marker distance range of 2 mm. In some examples, if the location information is outside a predetermined range (e.g., if the marker distance exceeds 50 mm), the system can forego generating and/or outputting a feedback signal. The number of audio tones (N) can be any integer greater than 1. In some examples, the processor can be initialized with a number of subtones between 2 and 50, such as 2, 3, 4, 5, 6, 10, 11, 12, 20, 21, 22, 30, 31, 32, 40, 41, 42, 49, or 50. In some examples, the processor can be initialized with a number of subtones larger than 50, such as 55, 60, 70, 80, 100, or the like.

In operation, the processor receives a new marker position input: <X, Y, Z> (e.g., relative to the detector). The marker's distance (x) may be calculated from the position vector: x=\sqrt{X²+Y²+Z²}, to be used as the scalar input into the audio tone calculations.

Each sub-tone activation may be calculated from the marker distance using the respective continuous activation function, in this example a gaussian PDF.

$${α}_{N} = {A}_{N} ( x ) = \frac{1}{\sqrt{2 π {σ}_{N}^{2}}} {e}^{- \frac{{( x - {μ}_{N} )}^{2}}{2 {σ}_{N}^{2}}}$$

Each sub-tone may be calculated by:

$${T}_{N} = {a}_{N} sin ( 2 π {tf}_{N} )$$

The net feedback waveform may be calculated as:

$${W}_{out} = \frac{1}{R} {\sum }_{1}^{N} {a}_{N} sin ( 2 π {tf}_{N} )$$

where R is the sum of all activation levels.

$$R = {\sum }_{1}^{N} {a}_{N}$$

Dividing the net waveform by R will normalize the signal to not exceed (−1 to +1), and to avoid signal clipping and speaker damage.

The processor may be in communication with and/or include a memory. The memory can be, for example, a random-access memory (RAM) (e.g., a dynamic RAM, a static RAM), a flash memory, a removable memory, and/or so forth. In some instances, instructions associated with performing the operations described herein (e.g., calculating a marker distance, generating sub-tones, etc.) can be stored within the memory and/or a storage medium (which, in some embodiments, includes a database in which the instructions are stored) and the instructions are executed at the processor.

In some instances, the processor includes one or more modules and/or components. Each module/component executed by the processor can be any combination of hardware-based module/component (e.g., a field-programmable gate array (FPGA), an application specific integrated circuit (ASIC), a digital signal processor (DSP), etc.), software-based module (e.g., a module of computer code stored in the memory and/or in the database, and/or executed at the processor, etc.), and/or a combination of hardware- and software-based modules. Each module/component executed by the processor is capable of performing one or more specific functions/operations as described herein. In some instances, the modules/components included and executed in the processor can be, for example, a process, application, virtual machine, and/or some other hardware or software module/component. The processor can be any suitable processor configured to run and/or execute those modules/components. The processor can be any suitable processing device configured to run and/or execute a set of instructions or code. For example, the processor can be a general-purpose processor, a central processing unit (CPU), an accelerated processing unit (APU), a field-programmable gate array (FPGA), an application specific integrated circuit (ASIC), a digital signal processor (DSP), and/or the like. Although the present disclosure has been described with reference to audio tones, the scope includes other embodiments which may be visual. For example, the sub-tones may be different colors which may have activation functions to cause each color to be displayed within a graphical user interface (GUI) for an overlapping width of the marker distance scale. Specifically, the same techniques may be used to generate a visual feedback signal, such as a polychromatic feedback signal. For example, an exemplary system can generate a visual feedback signal having a varying color over time. The varying color comprises a plurality of colors (e.g., a superimposition of colors), each color having a frequency different from the other color of the plurality of colors. Each color is associated with a respective activation function (of a plurality of activation functions) that modulates an amplitude of the respective color based on the obtained location information of the marker. In some examples, the plurality of colors are primary colors. In some examples, the number of colors may be any integer greater than 1, such as 2, 3, 4, or the like.

In one exemplary implementation, the processor is initialized with two colors: green (e.g., indicating a smaller distance or higher targeting precision) and red (e.g., indicating a larger distance or lower targeting precision). Accordingly, as the probe device is moved closer to the marker, the feedback signal transitions from a red color to intermediate colors generated according to some examples of the disclosure, then to a green color.

In one exemplary implementation, the processor is initialized with two colors: green (e.g., indicating a smaller distance or higher targeting precision) and white (e.g., indicating a larger distance or lower targeting precision). Accordingly, as the probe device is moved closer to the marker, the feedback signal transitions from a white color to intermediate colors generated according to some examples of the disclosure, then to a green color.

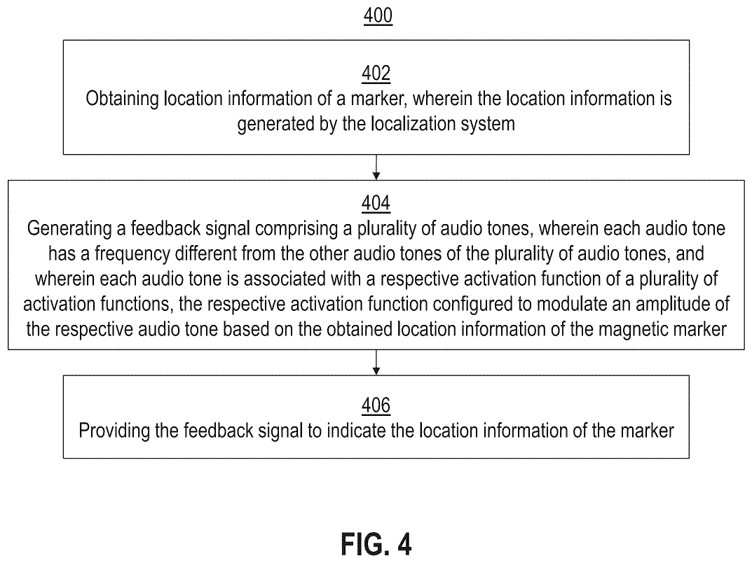

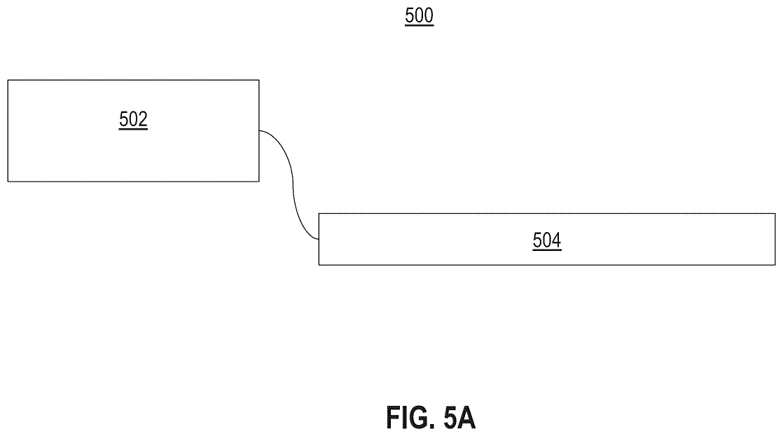

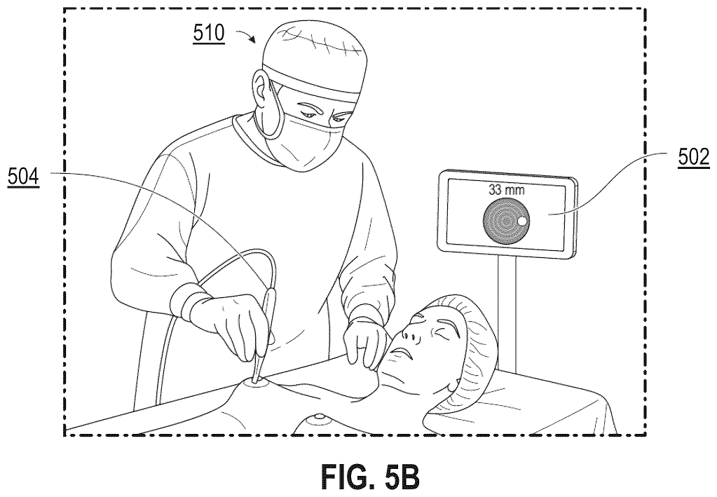

FIG. 4 illustrates an exemplary method for providing feedback by a localization system, in accordance with some examples. Process 400 is performed, for example, using one or more electronic devices of the localization system, such as devices 502 and/or 504 in FIG. 5 as described below. In some examples, process 400 may be performed using a client-server system, and the blocks of process 400 can be divided up in any manner between the server and one or more client devices. Thus, while portions of process 400 are described herein as being performed by particular devices, it will be appreciated that process 400 is not so limited. In process 400, some blocks are, optionally, combined, the order of some blocks is, optionally, changed, and some blocks are, optionally, omitted. In some examples, additional steps may be performed in combination with the process 400. Accordingly, the operations as illustrated (and described in greater detail below) are exemplary by nature and, as such, should not be viewed as limiting.

At block 402, an exemplary system (e.g., one or more electronic devices) obtains location information of a marker (e.g., x). The location information is generated by the localization system. The localization system can comprise a probe device (e.g., 504) comprising one or more magnetic sensors. The location information of the marker can be indicative of target precision of the probe device. In some examples, the location information comprises a component of a 3D position, a combination of multiple components of the 3D position, a distance, angular data, or any combination thereof.

At block 404, the system generates a feedback signal (e.g., Wₒᵤₜ) comprising a plurality of audio tones (e.g., {T}). Each audio tone has a frequency different from the other audio tones of the plurality of audio tones. For example, as shown in FIG. 2, each of the audio tones T has a different frequency.

Further, each audio tone is associated with a respective activation function that modulates an amplitude of the respective audio tone based on the obtained location information of the marker. In some examples, each activation function is a probability density function. A width of an activation function of the plurality of activation functions may be the same as or different from a width of another activation function of the plurality of activation functions. A peak amplitude of an activation function of the plurality of activation functions may be the same as or different from a peak amplitude of another activation function of the plurality of activation functions. Center frequencies of the plurality of activation functions are configured to be evenly spaced or varied.

At block 406, the system provides the feedback signal (e.g., Wₒᵤₜ) to indicate the location information of the marker, for example, by outputting the feedback signal via a speaker.

FIG. 5A illustrates an exemplary marker localization system, in accordance with some embodiments. The exemplary marker localization system 500 comprises an electronic device 502 and a probe device 504 comprising one or more magnetic sensors. The electronic device 502 and the probe 504 are communicatively coupled (e.g., via cable(s) or wirelessly) such that the probe 504 can transmit magnetic measurements acquired by the one or more magnetic sensors to the electronic device 502 for processing. In the depicted example, the electronic device 502 is located outside of a housing of the probe 504. In other examples, the electronic device 502 may be located within the housing of the probe 504. The electronic device 502 can comprise one or more processors for performing some or all of the steps of the process 300. The electronic device 502 may include or may be communicatively coupled to a display for displaying outputs associated with the process 300, as described below.

FIG. 5B depicts use of an exemplary marker localization system, in accordance with some embodiments. During a surgical procedure, a surgeon 510 can maneuver the probe 504 to detect the location of a marker implanted within a patient. The probe 504 comprises one or more magnetic sensors that generate magnetic field gradient values, which can be provided to the electronic device 502 for determining the location of the marker. In the depicted example, the electronic device 502 is a tablet computer with a display, which can display the estimated location of the marker on a graphical user interface. In addition, the electronic device 502 can perform some or all of the steps of the process 300 to detect magnetic noise, which may originate from various surgical instruments in the surgical environment. The outputs associated with the process 300 can be provided on the display of the electronic device 502.

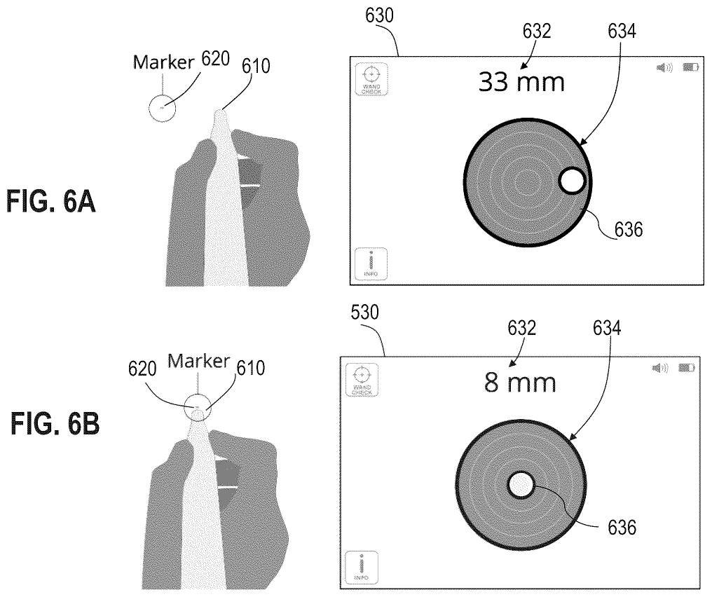

FIGS. 6A and 6B depict a probe tip 610 position (left) relative to a marker 620, and a corresponding result in a user interface 630 (right). In FIG. 6A, the probe tip 610 is to the right of the marker. The corresponding screen of the user interface 630 has a first indicator 632 showing a distance from the probe tip to the marker (33 mm) and a second indicator 634 showing the probe tip as a circle indicator to the right of a target center (“bullseye” target) thereby indicating the relative position. In FIG. 6B, the probe tip 610 is oriented directly above the marker 620 (i.e., the marker is in front of the probe tip). In the corresponding screen of the user interface 630, the first indicator 632 shows the distance from the probe tip to the marker (8 mm) and the second indicator 634 shows the probe tip circle at the center of the target.

In some examples, the color of the second indicator 634 may vary over time. The varying color comprises a plurality of colors (e.g., a superimposition of colors), each color having a frequency different from the other color of the plurality of colors. Each color is associated with a respective activation function that modulates an amplitude of the respective color based on the obtained location information of the marker. In some examples, the plurality of colors are primary colors. In some examples, the number of colors may be any integer greater than 1, such as 2, 3, 4, or the like.

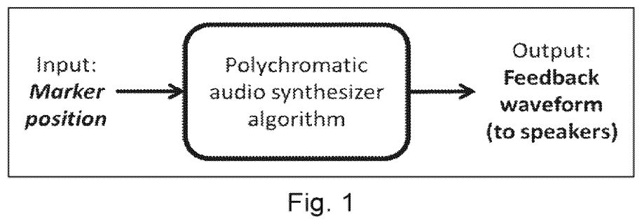

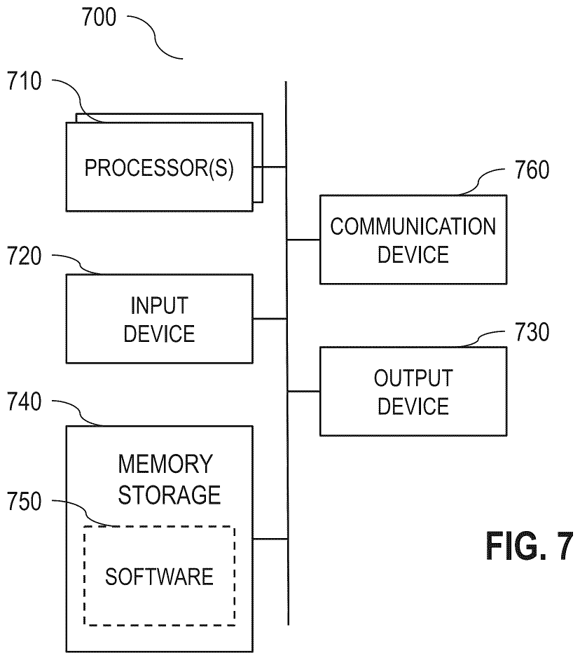

The operations described above with reference to FIGS. 1-4 are optionally implemented by components depicted in FIG. 7. It would be clear to a person having ordinary skill in the art how other processes are implemented based on the components depicted in FIG. 7.

FIG. 7 illustrates an example of a computing device in accordance with one embodiment. Device 700 can be a host computer connected to a network. Device 700 can be a client computer or a server. As shown in FIG. 7, device 700 can be any suitable type of microprocessor-based device, such as a personal computer, workstation, server or handheld computing device (portable electronic device) such as a phone or tablet. The device can include, for example, one or more of processor 710, input device 720, output device 730, storage 740, and communication device 760. Input device 720 and output device 730 can generally correspond to those described above, and can either be connectable or integrated with the computer.

Input device 720 can be any suitable device that provides input, such as a touch screen, keyboard or keypad, mouse, or voice-recognition device. Output device 730 can be any suitable device that provides output, such as a touch screen, haptics device, or speaker.

Storage 740 can be any suitable device that provides storage, such as an electrical, magnetic or optical memory including a RAM, cache, hard drive, or removable storage disk. Communication device 760 can include any suitable device capable of transmitting and receiving signals over a network, such as a network interface chip or device. The components of the computer can be connected in any suitable manner, such as via a physical bus or wirelessly.

Software 750, which can be stored in storage 740 and executed by processor 710, can include, for example, the programming that embodies the functionality of the present disclosure (e.g., as embodied in the devices as described above).

Software 750 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 740, that can contain or store programming for use by or in connection with an instruction execution system, apparatus, or device.

Software 750 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

Device 700 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 700 can implement any operating system suitable for operating on the network. Software 750 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example.

Although the present disclosure has been described with respect to one or more particular embodiments, it will be understood that other embodiments of the present disclosure may be made without departing from the spirit and scope of the present disclosure. The following are non-limiting sample claims intended solely to illustrate certain embodiments of the present disclosure.

What is claimed is:

1. A method for providing feedback by a localization system, comprising:
obtaining location information of a marker, wherein the location information is generated by the localization system;
generating a feedback signal comprising a plurality of audio tones, wherein each audio tone has a frequency different from the other audio tones of the plurality of audio tones, and wherein each audio tone is associated with a respective activation function of a plurality of activation functions, the respective activation function configured to modulate an amplitude of the respective audio tone based on the obtained location information of the marker; and
providing the feedback signal to indicate the location information of the marker.

2. The method of claim 1, wherein the localization system comprises a probe device comprising one or more magnetic sensors.

3. The method of claim 2, wherein the location information of the marker is indicative of target precision of the probe device.

4. The method of claim 1, wherein the location information comprises a component of a 3D position, a combination of multiple components of the 3D position, a distance, angular data, or any combination thereof.

5. The method of claim 1, wherein an activation function of the plurality of activation functions is a probability density function.

6. The method of claim 5, wherein a width of an activation function of the plurality of activation functions is the same as or different from a width of another activation function of the plurality of activation functions.

7. The method of claim 5, wherein a peak amplitude of an activation function of the plurality of activation functions is the same as or different from a peak amplitude of another activation function of the plurality of activation functions.

8. The method of claim 1, wherein center frequencies of the plurality of activation functions are configured to be evenly spaced or vary.

9. The method of claim 1, wherein providing the feedback signal comprises outputting the feedback signal via a speaker.

10. A system for providing feedback, comprising:
a localization system; and
a processor in electronic communication with the localization system, wherein the processor is configured to:
obtain location information of a marker, wherein the location information is generated by the localization system;
generate a feedback signal comprising a plurality of audio tones, wherein each audio tone has a frequency different from the other audio tones of the plurality of audio tones, and wherein each audio tone is associated with a respective activation function of a plurality of activation functions, the respective activation function configured to modulate an amplitude of the respective audio tone based on the obtained location information of the marker; and
provide the feedback signal to indicate the location information of the marker.

11. The system of claim 10, wherein the localization system comprises a probe device comprising one or more magnetic sensors.

12. The system of claim 11, wherein the location information of the marker is indicative of target precision of the probe device.

13. The system of claim 10, wherein the location information comprises a component of a 3D position, a combination of multiple components of the 3D position, a distance, angular data, or any combination thereof.

14. The system of claim 10, wherein an activation function of the plurality of activation functions is a probability density function.

15. The system of claim 14, wherein a width of an activation function of the plurality of activation functions is the same as or different from a width of another activation function of the plurality of activation functions.

16. The system of claim 14, wherein a peak amplitude of an activation function of the plurality of activation functions is the same as or different from a peak amplitude of another activation function of the plurality of activation functions.

17. The system of claim 10, wherein center frequencies of the plurality of activation functions are configured to be evenly spaced or vary.

18. The system of claim 10, wherein providing the feedback signal comprises outputting the feedback signal via a speaker.

19. A non-transitory computer-readable storage medium storing one or more programs for providing feedback by a localization system, the one or more programs comprising instructions, which when executed by one or more processors of an electronic device, cause the electronic device to:
obtain location information of a marker, wherein the location information is generated by the localization system;
generate a feedback signal comprising a plurality of audio tones, wherein each audio tone has a frequency different from the other audio tones of the plurality of audio tones, and wherein each audio tone is associated with a respective activation function of a plurality of activation functions, the respective activation function configured to modulate an amplitude of the respective audio tone based on the obtained location information of the marker; and
provide the feedback signal to indicate the location information of the marker.
